(19) United States
(12) Patent Application Publication
(10) Pub. No.: US 2018/0020808 A1
(43) Pub. Date: Jan. 25, 2018

(54) TELESCOPING CONTAINER FOR LIQUID COSMETIC PRODUCT

(71) Applicant: HCT GROUP HOLDINGS LIMITED, CENTRAL (CN)

(72) Inventors: ARMANDO VILLARREAL, LOS ANGELES, CA (US); STEPHEN CHARLES BURDITT, LOS ANGELES, CA (US)

(73) Assignee: HCT GROUP HOLDINGS LIMITED, CENTRAL (CN)

(21) Appl. No.: 15/654,170

(22) Filed: Jul. 19, 2017

Related U.S. Application Data
(60) Provisional application No. 62/365,835, filed on Jul. 22, 2016.

(51) Int. Cl. A45D 34/04; A61Q 1/02; A61K 8/04

(52) U.S. Cl. CPC A45D 34/043; A61K 8/04; A61Q 1/02; A45D 34/06

(57) ABSTRACT

A device for dispensing a cosmetic product may include a base, a screw, a piston, a sealing member, and a body. The screw and piston are threadingly engaged such that rotation of the body and base in opposite directions results in the screw and piston being moved upwards into the body, driving the cosmetic product out through an opening in an upper surface of the body.

### CROSS REFERENCE TO RELATED APPLICATIONS

The present application claims the benefit of and priority to U.S. Provisional Patent Application Ser. No. 62/365,835, filed on Jul. 22, 2016, titled TELESCOPING CONTAINER FOR LIQUID COSMETIC PRODUCT, the disclosure of which is incorporated herein by reference.

### TECHNICAL FIELD

The present disclosure relates generally to a cosmetic applicator which has a twist up mechanism for dispensing cosmetic product.

### SUMMARY

According to some embodiments of the present disclosure, a device for dispensing a cosmetic product includes a base having an interior space, a screw having at least one external thread and at least one internal thread, the screw disposed within the interior space of the base, the internal thread threadingly engaged with one or more protrusions on the base, a piston having an inner cavity and one or more projections extending into the inner cavity, wherein the projections on the inner cavity of the piston are threadingly engaged with the external thread on the screw, the piston configured to be disposed over the screw within the interior space of the base, a sealing member configured to fit over an upper end of the piston, the sealing member having an oval upper surface, and a body with an open lower end configured to engage an upper end of the base, the body having an inner wall defining a product container, the inner wall having an oval cross section taken perpendicular to a longitudinal axis of the device, the oval cross section configured to receive the oval upper surface of the sealing member in a slidable friction fit. Rotation of the base relative to the body causes the screw to rotate, thereby moving the piston and sealing member upwards through the body.

Alternatively or additionally, in another example, the base includes a central insert, wherein the interior space is defined between an inner wall of the base and an outer wall of the central insert.

Alternatively or additionally, in another example, the one or more protrusions on the base are disposed on the outer wall of the central insert.

Alternatively or additionally, in another example, the one or more protrusions on the base are disposed adjacent an upper end of the central insert.

Alternatively or additionally, in another example, the sealing member has a cylindrical lower surface configured to fit over a cylindrical upper surface of the piston.

Alternatively or additionally, in another example, the oval upper surface of the sealing member is flexible.

Alternatively or additionally, in another example, the device further comprises a collar connected to an upper open end of the body, the collar having an opening therein for dispensing product.

Alternatively or additionally, in another example, the device further comprises a cap configured to be connected to the collar.

Alternatively or additionally, in another example, the body is transparent.

In another example, a device for dispensing a cosmetic product includes a container configured to hold a volume of cosmetic product, the container having an inner surface with an oval cross section taken perpendicular to a longitudinal axis of the container, and a cosmetic dispensing system coupled to an open lower end of the container. The cosmetic dispensing system includes a base, a hollow screw disposed inside the base, the screw having at least one internal thread and at least one external thread, wherein the at least one internal thread is threadingly engaged with one or more protrusions on the base, a piston configured to fit over the screw and within the base, the piston having one or more projections threadingly engaged with the at least one external thread on the screw, and a sealing member attached to the piston, the sealing member having an oval sealing surface configured to frictionally engage the inner oval surface of the container. The dispensing system is configured such that rotation of the base relative to the container rotates the screw which moves the piston and attached sealing member upwards through the container.

Alternatively or additionally, in another example, the base includes a central insert, wherein the one or more protrusions on the base are disposed on an outer wall of the central insert.

Alternatively or additionally, in another example, the one or more protrusions on the base include first and second protrusions disposed opposite one another adjacent an upper end of the central insert.

Alternatively or additionally, in another example, the one or more projections on the piston include first and second projections disposed opposite one another on a lower end of an inner surface of the piston.

Alternatively or additionally, in another example, the screw includes first and second internal threads and first and second external threads, wherein the first and second projections on the piston are threadingly engaged with the first and second external threads, respectively, and the first and second protrusions on the base are threadingly engaged with the first and second internal threads, respectively.

Alternatively or additionally, in another example, the base and the container have cylindrical outer surfaces.

Alternatively or additionally, in another example, the sealing member has a cylindrical lower surface configured to fit over a cylindrical upper surface of the piston.

Alternatively or additionally, in another example, the oval sealing surface of the sealing member is flexible.

Alternatively or additionally, in another example, the device further comprises a collar connected to an upper open end of the container, the collar having an opening therein for dispensing product.

Alternatively or additionally, in another example, the container is transparent.

In another example, a device for dispensing a cosmetic product comprises a base having an interior space, a screw having at least one external thread, and an inner surface having at least one protrusion thereon, the screw disposed within the interior space of the base, a piston having at least one external thread configured to threadingly engage the at least one protrusion on the inner surface of the screw, the piston configured to be disposed within the screw, a sealing member connected to the piston, the sealing member having an oval upper surface, and a body with an open lower end configured to engage an upper end of the base, the body having an inner wall defining a product container, the inner wall having an oval cross section taken perpendicular to a longitudinal axis of the device, the oval cross section configured to receive the oval upper surface of the sealing member in a slidable friction fit, the body having at least one projection on the inner wall, the at least one projection configured to threadingly engage the external thread on the screw. Rotation of the body relative to the base causes the screw to rotate, thereby moving the piston and sealing member upwards through the body.

### BRIEF DESCRIPTION OF THE DRAWINGS

The foregoing and other aspects of the present disclosure are best understood from the following detailed description when read in connection with the accompanying drawings. In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a device for dispensing a cosmetic product according to one example, with the cap removed,

FIG. 2 is a side exploded view of the device of FIG. 1 including the cap,

FIG. 3 is a side cross-sectional view of the device of FIG. 1,

FIG. 4 is a perspective exploded view of the cosmetic dispensing system of the device of FIG. 1,

FIG. 5 is a perspective view of the screw of the device of FIG. 1,

FIG. 6 is a perspective view of the piston of the device of FIG. 1,

FIG. 7 is a top view of the body of FIG. 1,

FIG. 8 is a bottom view of the sealing member of FIG. 1,

FIG. 9 is a perspective exploded view of a device for dispensing a cosmetic product according to another example, and

FIG. 10 is a cross-sectional view of the device of FIG. 9.

### DETAILED DESCRIPTION

The present disclosure relates generally to a device which dispenses a cosmetic product. This technology is particularly well-suited for, but by no means limited to, liquid or cream cosmetic products such as concealer, foundation, lotion, or serum.

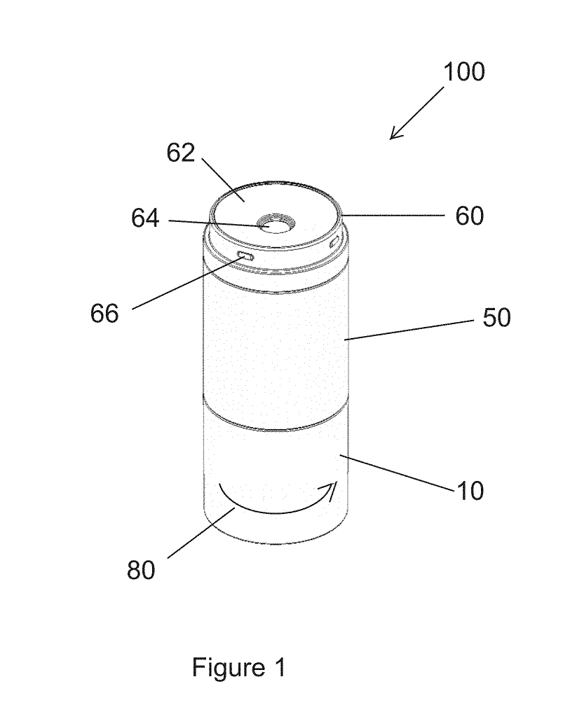

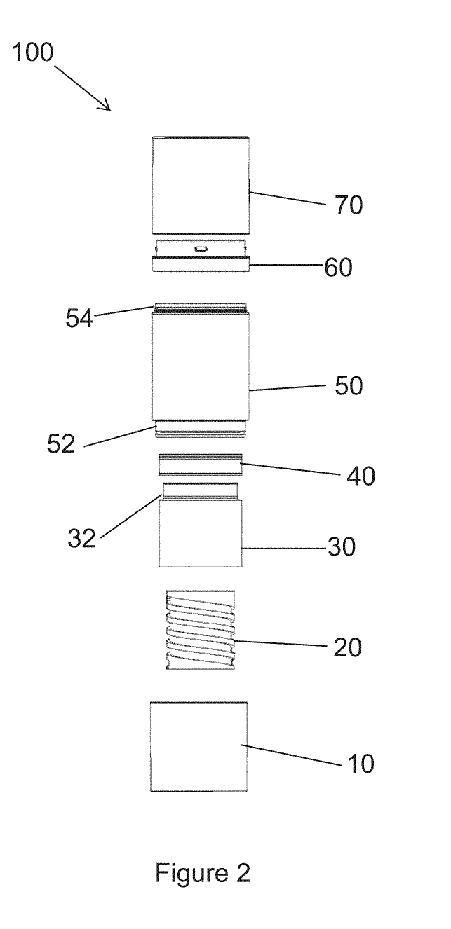

FIG. 1 illustrates a device 100 for dispensing a cosmetic product such as a liquid or cream. The device 100 includes a base 10 containing an actuating mechanism that expels the product through the opening 64, a container or body 50 for holding the cosmetic product, and a collar 60 providing the opening 64 through which the product is dispensed. In some examples, the opening may include a sealing member (not shown) such as a membrane with a slit therein to prevent liquid product from leaking out if the device 100 is knocked over or put down on its side without the cap. The collar 60 includes an upper face 62 which may be angled towards the opening 64 to allow excess product to flow back into the body 50. The body 50 may include a separate internal reservoir (not shown). The collar 60 is disposed over the upper open end of the body 50, and may include connection elements 66 to connect a cap 70 (shown in FIG. 2) to the body 50. The collar 60 may be fixed to the body 50 or the collar 60 may be removable to permit re-filling the device. The cap 70 may engage the collar 60 with a snap fit, threaded connection, friction fit, or any other suitable connection. The components of the device 100 may be made of plastics or other materials as noted herein. In some examples, the body 50 may be transparent plastic or glass.

The device 100 for dispensing a cosmetic product is shown in an elongated cylindrical configuration. However, it should be understood that examples of the disclosure described herein may be applied to various other configurations in other examples. For example, the device 100 may alternatively be embodied in an elongated cubical configuration where the components of the device are all cubical in shape. Other shapes include oval, triangular, heart shaped, etc.

FIG. 2 shows all of the elements in the device 100. The base 10 receives therein an actuating mechanism including a screw 20, a piston 30, and a sealing member 40. The body 50 is attached to the base 10, and the collar 60 is attached to the body 50. The cap 70 is removably connectable to the collar 60. The body 50 is rotatably attached to the base 10. The body 50 may include a reduced diameter upper portion 54 that receives the collar 60 thereon. The body 50 and the collar 60 may be fixed together or the collar may be removable. The body 50 may include a reduced diameter lower portion 52 that fits inside the upper opening of the base 10. The reduced diameter lower portion 52 may engage the inner wall of the base 10 in a freely rotational engagement. For example, the reduced diameter lower portion 52 may include a recess configured to receive a raised ridge on the inner wall of the base 10. In another example, the reduced diameter lower portion 52 may include one or more raised ridges that engage one or more recesses or grooves on the inner wall of the base 10.

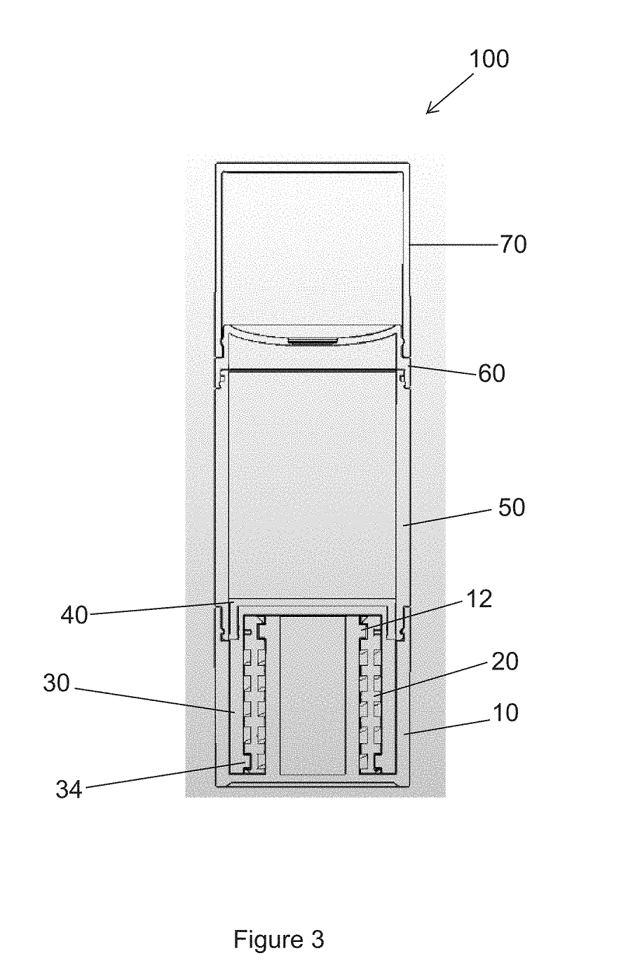

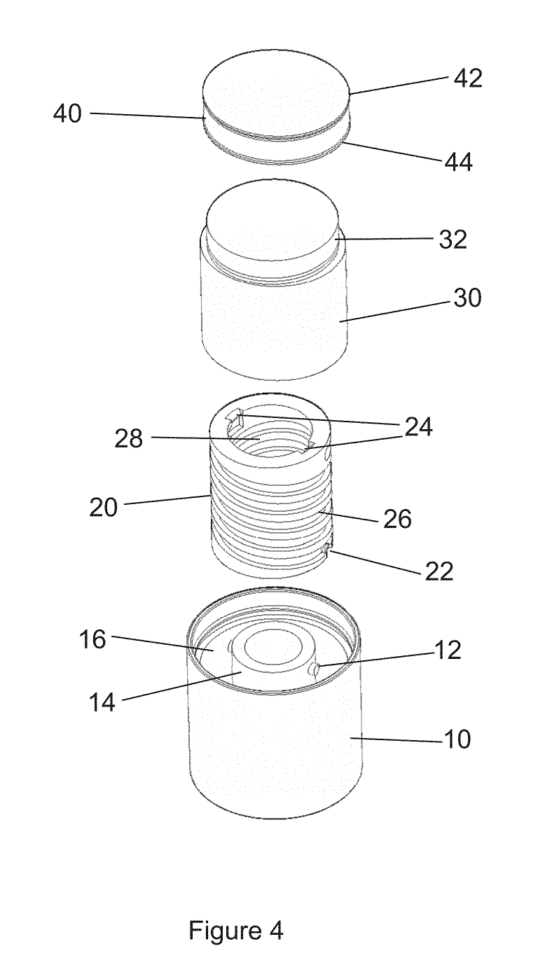

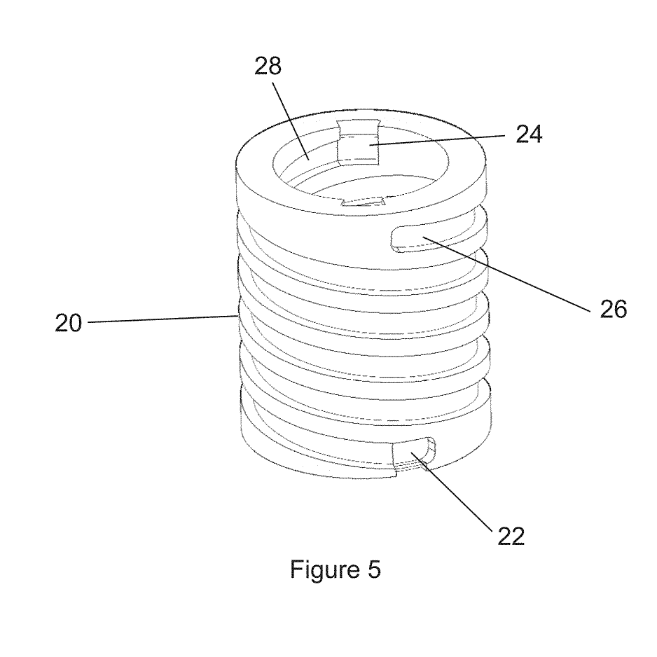

The details of the actuating mechanism are shown in FIGS. 3-6. The actuating mechanism provides a screw-type movement to advance the sealing member 40 through the body 50. The base 10, screw 20, and piston 30 are movably interconnected. The base 10 has a solid bottom, an open top, and an interior space 16 defined between an inner wall of the base 10 and an outer wall of a central column or insert 14. The interior space 16 is sized and configured to receive the screw 20 and piston 30, as seen in FIG. 3. The outer surface of the central insert 14 of the base 10 may have one or more protrusions 12. In the example shown in FIGS. 3 and 4, the base 10 has two protrusions 12 spaced apart on opposite sides of the central insert 14. The protrusions are sized and configured to engage recesses 24 on an inner wall of the screw 20. The screw 20 may be a hollow cylinder. The recesses 24 define upper end stops of internal threads 28 extending along the inner surface of the screw 20, as shown in FIG. 5. When the screw 20 is disposed within the interior space 16 of the base 10, the protrusions 12 slide along the internal threads 28, causing the screw 20 to rise upward out of the base.

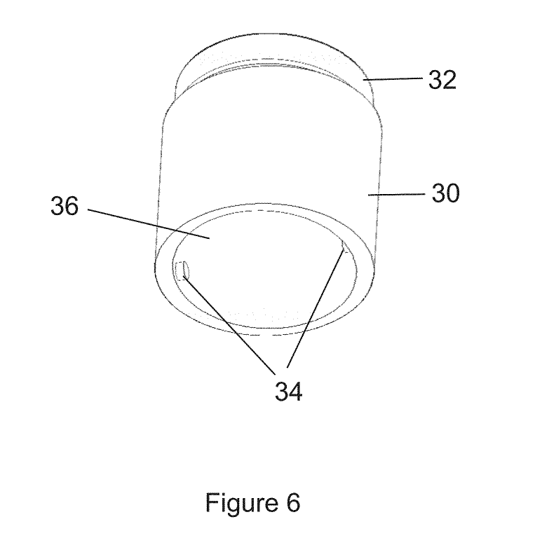

The screw 20 has external threads 26 ending with recesses 22 sized and configured to receive projections 34 extending from the inner wall of the piston 30. See FIGS. 5 and 6. The piston 30 has an inner cavity 36 and an open bottom. Projections 34 extend from the inner wall into the inner cavity 36. In the example illustrated, two opposing projections 34 reside on the inner wall of the piston 30. The upper end of the piston 30 may include a reduced diameter region 32 and a closed top. The reduced diameter region 32 is sized and configured to receive the sealing member 40. The piston 30 fits over the screw 20 with the projections 34 sliding along the external threads 26. When the device 100 is filled with product, and before use, the screw 20 and piston 30 are positioned entirely within the base 10, with the protrusions 12 on the base 10 disposed at the upper end of the internal threads 28 on the screw 20, and the projections 34 on the piston 30 disposed at the lower end of the external threads 26 on the screw 20. See FIG. 3.

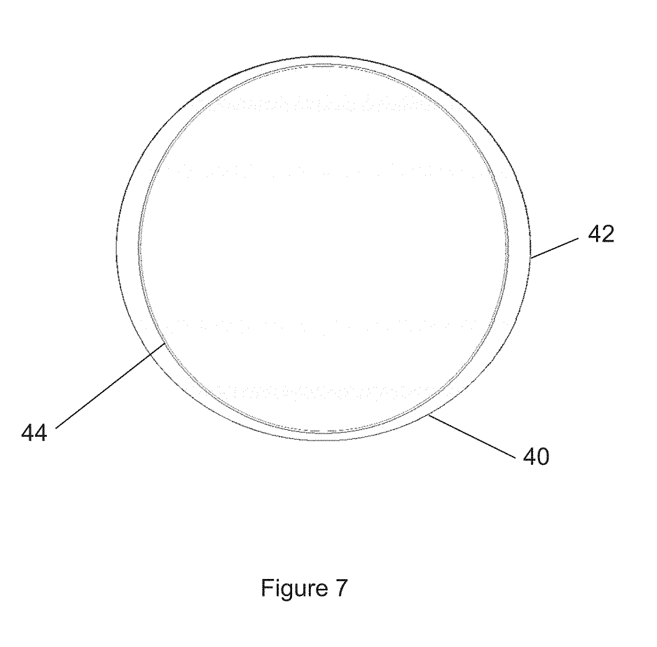

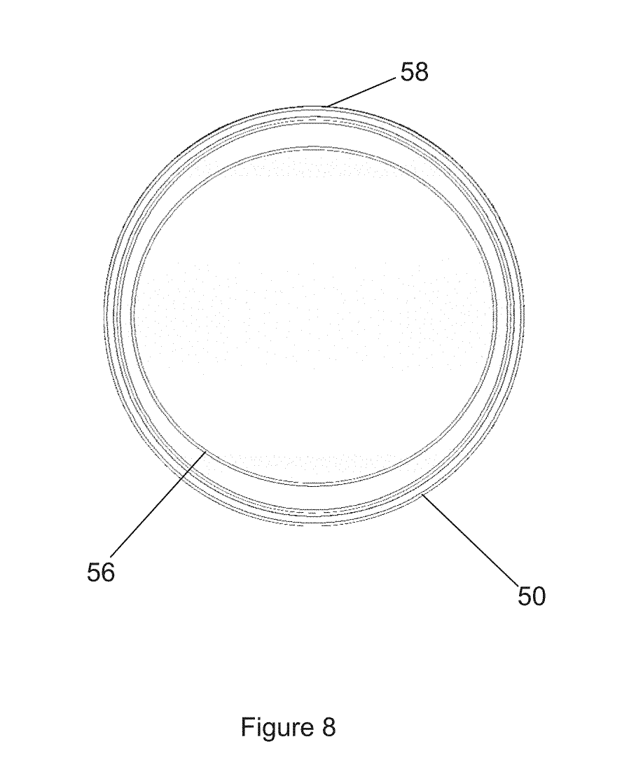

The reduced diameter region 32 of the piston 30 may be cylindrical and is sized to receive a lower portion 44 of the sealing member 40 thereon. The sealing member 40 may have an inner chamber configured to receive the reduced diameter region 32 of the piston 30. The sealing member 40 is fixed to the piston. The sealing member 40 has an upper surface 42 that is perpendicular to the longitudinal axis of the device. The upper surface 42 has an oval shape with an outer dimension that is larger than the outer dimension of the lower portion 44. As seen in FIG. 7, which shows the sealing member 40 viewed from the bottom, the upper surface 42 is an oval and extends beyond the outer edge of the lower portion 44. The upper surface 42 is flexible and may be made of a resilient material such as rubber or silicone. The upper surface 42 is sized and configured to form a seal with an inner surface 56 of the body 50. As seen in FIG. 8, the inner surface 56 of the body 50 is also oval as compared to the exterior wall 58, which is illustrated as being circular. The oval upper surface 42 of the sealing member 40 and the oval inner surface 56 of the body 50 combine to prevent the sealing member 40 from rotating within the body 50. The sealing member 40 moves axially upwards through the body, pushing product up and out through the opening 64.

The actuation of the screw assembly is as follows. The user holds the body 50 and rotates the base 10 relative to the body 50. For example, the base 10 may be rotated in a counter-clockwise direction as shown by arrow 80 in FIG. 1. Rotation of the base 10 causes the protrusions 12 to travel along the internal threads 28 from the upper end of the screw towards the lower end, thereby moving the screw 20 towards and into the body 50. As the screw 20 is moved upwards, the projections 34 on the piston travel up the external threads 26. Because the upper surface 42 of the sealing member 40 is oval and disposed within the oval inner body chamber, the sealing member 40 and the piston 30 it is attached to cannot rotate. The sliding engagement of the projections 34 in the external threads 26 causes rotational movement of the screw 20 to be translated into upward movement of the piston 30. Rotation of the base 10 relative to the body 50 thus causes the sealing member 40 to travel upwards through the body 50, thereby dispensing product.

The dual threaded screw 20, base 10, and piston 30 allow for a greater range of movement of the sealing member 40 from a lower position when the device is filled with product, to an upper position when the product has been fully dispensed. The threaded actuation mechanism takes up less volume in the entire device than conventional screw type mechanisms, allowing for a greater volume of product to be presented in a smaller container. Additionally, in examples where the body 50 is transparent, the screw 20 is not visible to the user. Only the solid walls of the piston 30 and sealing member 40 are viewable through the transparent body 50 as the product is dispensed.

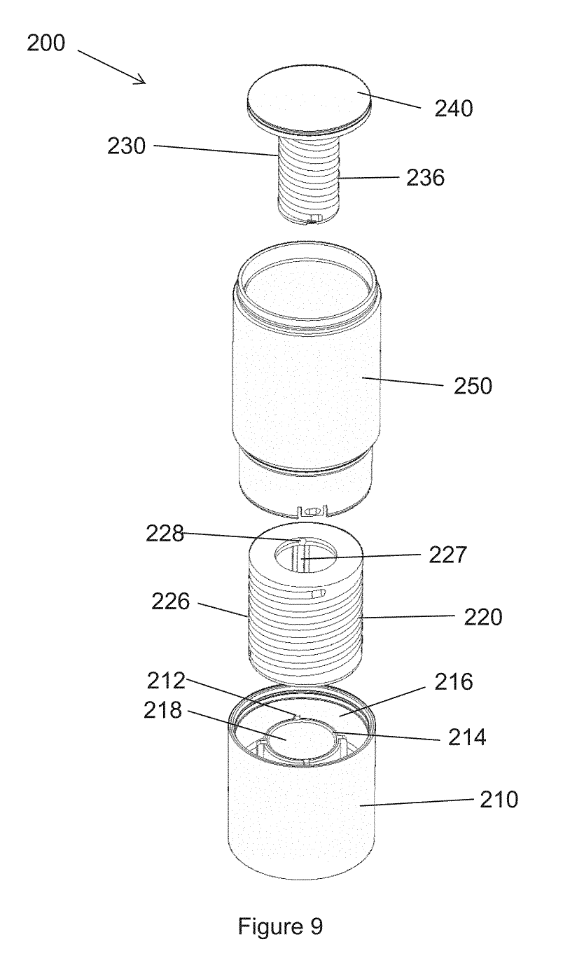

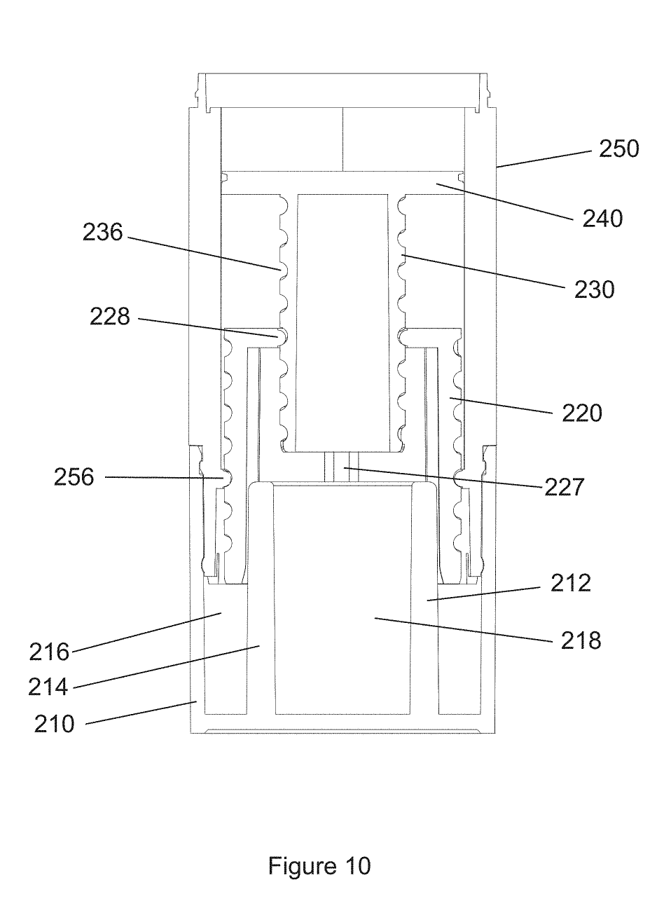

Another embodiment of actuating mechanism is shown in FIGS. 9 and 10. In device 200, the collar and cap are not shown and may be the same as for device 100. The actuating mechanism provides a screw-type movement to advance a sealing member through the body 250. The base 210, screw 220, and piston 230 are movably interconnected. The freely rotatable connection between the base 210 and the body 250 is the same as described above for device 100. The base 210 has a solid bottom, an open top, and an interior space 216 defined between an inner wall of the base 210 and an outer wall of a central column or insert 214. The insert 214 has an interior cavity 218 sized and configured to receive the threaded portion of the piston 230. The interior space 216 of the base 210 is sized and configured to receive the screw 220, as seen in FIG. 10. The screw 220 has one or more external thread 226 but no internal thread. The inner wall of the screw 220 has one or more slot 227 sized and configured to receive one or more elongated ridges 212. The device 200 as shown in FIG. 9 has a base 210 with four ridges 212 spaced apart equally around the insert 214. The screw 220 has four slots 227 at corresponding positions around the inner surface of the screw 220. The external thread 226 on the screw 220 engages one or more bump 256 on an inner wall of the body 250. The bump 256 is sized and configured to slide along the external thread 226, moving the screw 220 upwards relative to the body 250. In the example shown in FIG. 10, the body 250 has two bumps 256 on opposite sides of the body. When the screw 220 is disposed within the interior space 216 of the base 210, the bump 256 may be disposed in an upper portion of the external thread 226.

The piston 230 is sized and configured to be received within an interior of the screw 220 and the interior cavity 218 of the base 210. The piston 230 has an external thread 236 sized and configured to receive one or more projection 228 extending from the inner wall of the screw 220. In the example shown in FIG. 10, the screw 220 has two projections 228 on opposite sides of the screw 220. The piston 230 may have an integrated sealing member 240, as shown in FIG. 9. In other examples, the piston 230 may have a reduced diameter portion that receives a separate sealing member, as discussed above with regard to device 100. Similar to the example shown in FIGS. 1-8, the sealing member 240 has an upper surface that is oval in shape and is sized and configured to form a seal with an inner surface of the body 250 that also has an oval shape in cross-section. The piston 230 fits with the interior of the screw 220 with the projection 228 on the screw 220 sliding along the external threads 236 of the piston 230. When the device 200 is filled with product, and before use, the screw 220 and piston 230 are positioned entirely within the base 210, with the bump 256 on the body 250 disposed at the upper end of the external threads 226 on the screw 220, and the projection 228 on the interior of the screw 220 disposed at the upper end of the external threads 236 on the piston 230. FIG. 10 shows the device with the screw 220 and piston 230 partially extended, in a configuration that will be achieved when a majority of the product has been expelled.

Actuation of the device 200 is achieved by turning the body 250 relative to the base 210. As the body 250 is rotated, the bump 256 on the body travels down the external thread 226 on the screw 220, moving the screw 220 upwards into the body 250. The sliding engagement of the ridges 212 on the base 210 in the slots 227 of the screw 220 prevent the screw 220 from rotating, allowing only axial movement of the screw 200. Upward movement of the screw 220 causes the projection 228 on the interior of the screw 220 to travel down the external thread 236 on the piston 230, thereby moving the piston 230 upwards through the body 250, resulting in product being expelled from the device.

Each of the above non-limiting examples can stand on its own, or the features and elements of one example may be combined in various permutations or combinations with one or more features and elements of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for dispensing a cosmetic product, the device comprising:
a base having an interior space;
a screw having at least one external thread and at least one internal thread, the screw disposed within the interior space of the base, the internal thread threadingly engaged with one or more protrusions on the base;
a piston having an inner cavity and one or more projections extending into the inner cavity, wherein the projections on the inner cavity of the piston are threadingly engaged with the external thread on the screw, the piston configured to be disposed over the screw within the interior space of the base;
a sealing member configured to fit over an upper end of the piston, the sealing member having an oval upper surface; and
a body with an open lower end configured to engage an upper end of the base, the body having an inner wall defining a product container, the inner wall having an oval cross section taken perpendicular to a longitudinal axis of the device, the oval cross section configured to receive the oval upper surface of the sealing member in a slidable friction fit;
wherein rotation of the base relative to the body causes the screw to rotate, thereby moving the piston and sealing member upwards through the body.

2. The device of claim 1, wherein the base includes a central insert, wherein the interior space is defined between an inner wall of the base and an outer wall of the central insert.

3. The device of claim 2, wherein the one or more protrusions on the base are disposed on the outer wall of the central insert.

4. The device of claim 3, wherein the one or more protrusions on the base are disposed adjacent an upper end of the central insert.

5. The device of claim 1, wherein the sealing member has a cylindrical lower surface configured to fit over a cylindrical upper surface of the piston.

6. The device of claim 5, wherein the oval upper surface of the sealing member is flexible.

7. The device of claim 1, further comprising a collar connected to an upper open end of the body, the collar having an opening therein for dispensing product.

8. The device of claim 7, further comprising a cap configured to be connected to the collar.

9. The device of claim 1, wherein the body is transparent.

10. A device for dispensing a cosmetic product, the device comprising:
a container configured to hold a volume of cosmetic product, the container having an inner surface with an oval cross section taken perpendicular to a longitudinal axis of the container; and
a cosmetic dispensing system coupled to an open lower end of the container and comprising:
a base;
a hollow screw disposed inside the base, the screw having at least one internal thread and at least one external thread, wherein the at least one internal thread is threadingly engaged with one or more protrusions on the base;
a piston configured to fit over the screw and within the base, the piston having one or more projections threadingly engaged with the at least one external thread on the screw; and
a sealing member attached to the piston, the sealing member having an oval sealing surface configured to frictionally engage the inner oval surface of the container;
wherein the dispensing system is configured such that rotation of the base relative to the container rotates the screw which moves the piston and attached sealing member upwards through the container.

11. The device of claim 10, wherein the base includes a central insert, wherein the one or more protrusions on the base are disposed on an outer wall of the central insert.

12. The device of claim 11, wherein the one or more protrusions on the base include first and second protrusions disposed opposite one another adjacent an upper end of the central insert.

13. The device of claim 12, wherein the one or more projections on the piston include first and second projections disposed opposite one another on a lower end of an inner surface of the piston.

14. The device of claim 13, wherein the screw includes first and second internal threads and first and second external threads, wherein the first and second projections on the piston are threadingly engaged with the first and second external threads, respectively, and the first and second protrusions on the base are threadingly engaged with the first and second internal threads, respectively.

15. The device of claim 10, wherein the base and the container have cylindrical outer surfaces.

16. The device of claim 15, wherein the sealing member has a cylindrical lower surface configured to fit over a cylindrical upper surface of the piston.

17. The device of claim 10, wherein the oval sealing surface of the sealing member is flexible.

18. The device of claim 10, further comprising a collar connected to an upper open end of the container, the collar having an opening therein for dispensing product.

19. The device of claim 10, wherein the container is transparent.

20. A device for dispensing a cosmetic product, the device comprising:
a base having an interior space;
a screw having at least one external thread, and an inner surface having at least one protrusion thereon, the screw disposed within the interior space of the base;
a piston having at least one external thread configured to threadingly engage the at least one protrusion on the inner surface of the screw, the piston configured to be disposed within the screw;
a sealing member connected to the piston, the sealing member having an oval upper surface; and
a body with an open lower end configured to engage an upper end of the base, the body having an inner wall defining a product container, the inner wall having an oval cross section taken perpendicular to a longitudinal axis of the device, the oval cross section configured to receive the oval upper surface of the sealing member in a slidable friction fit, the body having at least one projection on the inner wall, the at least one projection configured to threadingly engage the external thread on the screw;
wherein rotation of the body relative to the base causes the screw to move axially upwards into the body, thereby moving the piston and sealing member upwards through the body.
